(19) United States
(12) Patent Application Publication
(10) Pub. No.: US 2021/0129406 A1
(43) Pub. Date: May 6, 2021

(54) DRUG SOLVATES IN THERMAL PROCESSES TO MAKE SOLID DISPERSIONS AT LOWER PROCESSING TEMPERATURES

(71) Applicant: BOARD OF REGENTS, THE UNIVERSITY OF TEXAS SYSTEM, Austin, TX (US)

(72) Inventors: Robert O. WILLIAMS, III, Austin, TX (US); Xiangyu MA, Austin, TX (US); Siyuan HUANG, Indianapolis, IN (US)

(21) Appl. No.: 17/088,680

(22) Filed: Nov. 4, 2020

Related U.S. Application Data
(60) Provisional application No. 62/930,321, filed on Nov. 4, 2019.

(51) Int. Cl. B29C 48/00; A61K 31/55; B29C 48/285; B29C 48/505; B29C 48/92; B29C 48/88; B29C 48/85; A61K 47/34; A61K 47/22

(52) U.S. Cl. CPC B29C 48/022; A61K 31/55; B29C 48/286; B29C 48/505; A61K 47/22; B29C 48/911; B29C 48/85; A61K 47/34; B29C 48/92

(57) ABSTRACT

Methods of producing substantially amorphous pharmaceutical formulations by hot melt extrusion (HME) are provided. In some aspects, the methods can be performed at lower temperatures than are typically required for HME. Pharmaceutical formations produced by these methods are also provided.

This application claims the benefit of priority to U.S. Provisional Application Ser. No. 62/930,321, filed on Nov. 4, 2019, the entire contents of which are hereby incorporated by reference.

### BACKGROUND

### 1. Field

The present invention relates generally to the field of pharmaceuticals and pharmaceutical manufacture. More particularly, it concerns compositions and methods of preparing a drug composition.

### 2. Description of Related Art

Poorly-water soluble drugs represent a significant portion of the molecular entities in the industry's drug development pipeline. While approximately 40% of the current commercial products are poorly soluble based on the biopharmaceutical classification system (BCS), and an estimated 90% of compounds in development can be classified as poorly soluble.

The strategy of using amorphous solid dispersions (SD) enables the delivery and improves the dissolution/bioavailability of poorly water-soluble drugs. SDs have gained popularity in the pharmaceutical industry that 30 amorphous/nanocrystalline SDs based products have been approved by the Food and Drug Administration (FDA) in the last two decades, and SDs will continue to play an important role in developing poorly water-soluble drugs.

Thermal processes, like hot melt extrusion (HME), have been used to prepare solid dispersions for solubility enhancement purposes. The energy generated by the extruder in the form of mechanical and thermal output enables the dispersion and dissolution of crystalline drugs in polymeric carriers. However, due to the narrow processing window present in current approaches, extrusion cannot be applied to many drugs to prepare solid dispersions due to the degradation of drug molecules/polymers. Therefore, there exists a need for improved methods that extend the processing window and employ less harsh conditions in the preparation of various drugs as solid dispersions.

However, more than one-third of pharmaceutical compounds are known to exist as hydrates. Drug hydrates/solvates exhibit different physicochemical properties compared to the anhydrous/nonsolvate drug forms, and these differences can contribute to significant changes in extrusion. Dehydration/desolvation of such compounds can happen during extrusion and create disordered molecular structure, which may facilitate the preparation of solid dispersions. Also, dehydration/desolvation may occur at a mild condition such as lower temperature compared to the melting of compounds. Therefore, by using drug hydrates/solvates in extrusion, the process conditions can be significantly eased, and the processing window can be greatly extended to be able to prepare various drugs as solid dispersions.

### SUMMARY OF THE INVENTION

In some embodiments, the present disclosure provides methods producing a pharmaceutical formulation comprising: (a) providing starting material comprising an active pharmaceutical ingredient (API) solvate; and (b) processing the API solvate by hot melt extrusion (HME) along with at least a first polymer thereby producing a pharmaceutical formulation of the API ansolvate, wherein the HME processing is performed at a temperature that is at least 10% less than the temperature required for processing the API ansolvate as the starting material.

In other embodiments, the present disclosure provides methods producing a pharmaceutical formulation comprising: (a) providing starting material comprising an active pharmaceutical ingredient (API) solvate; and (b) processing the API solvate by hot melt extrusion (HME) along with at least a first polymer thereby producing a pharmaceutical formulation of an API ansolvate, wherein the HME processing is performed at a temperature of less than about 120° C.

In some aspects, the API solvate is an API hydrate. In some aspects, wherein the pharmaceutical formulation is substantially amorphous. In some aspects, the HME is performed using a single screw extruder device. In other aspects, the HME is performed using a twin screw extruder device. In some aspects, the screws of the twin screw extruder device are counter rotating. In other aspects, the screws of the twin screw extruder device are co-rotating. In some aspects, the HME is performed at screw speed of about 50 to 800 rpm. In further aspects, HME is performed at screw speed of about 50 to 300, 100-300, or 100-200 rpm. In some aspects, the HME is performed using an extruder device having a barrel length/diameter (L/D) ratio of about 20-40:1. In some aspects, the HME processing is performed at a temperature that is at least 15%, 20%, 25%, 30%, 35%, 40%, 45%, 50%, 55% or 60% less than the temperature required for processing an anhydrous form of the API as the starting material. In some aspects, the HME processing is performed at a temperature that is at least 10° C., 15° C., 20° C., 25° C., 30° C., 35° C., 40° C., 45° C., 50° C., 55° C. or 60° C. less than the temperature required for processing an anhydrous form of the API as the starting material. In some aspects, the HME processing is performed at a temperature of less than 120° C. In some aspects, the HME processing is performed at a temperature of less than 100° C. In some aspects, the HME processing is performed at a temperature of less than 80° C.

In some aspects, the API solvate is a monosolvate or disolvate. In other aspects, API solvate is a tri-, tetra- or penta-solvate. In some aspects, the API solvate is a monohydrate. In other aspects, the API solvate is a dihydrate. In still other aspects, the API solvate is a tri-, tetra- or penta-hydrate. In some aspects, the hydrated form of the API solvate has the formula API.XH₂O, wherein X is 1-10. In some aspects, the API solvate is a mixture of different API solvates. In some aspects, the API solvate is a mixture of API forms with different hydration levels. In some aspects, the API is present in the pharmaceutical formulation in an amount of from about 0.5% to 20%. In further aspects, the API is present in the pharmaceutical formulation in an amount of from about 1% to 15%, 5% to 15%, 7% to 12%, or about 10%. In some aspects, the first polymer comprises a polyvinyl caprolactam-polyvinyl acetate polyethylene glycol graft copolymer. In some aspects, the pharmaceutical formulation comprises from about 1% to about 90% of the polymer. In further aspects, the pharmaceutical formulation comprises from about 10% to about 90%, about 20% to about 90%, about 30% to about 80%, about 40% to about 70% or about 50% to about 70% of the polymer.

In some aspects, the methods further comprise processing the API by HME along with at least a first polymer and at least a first plasticizer or excipient. In some aspects, the plasticizer is present in in an amount of about 10%-50% w/w. In further aspects, the plasticizer is present in in an amount of about 10%-40%, 20%-50%, or 20%-40% w/w. In some aspects, the first plasticizer comprises vitamin E succinate, propylene glycol or polyethylene glycol. In some aspects, the API comprises carbamazepine (CBZ). In further aspects, the hydrated form of the API solvate comprises carbamazepine (CBZ) dihydrate. In some aspects, the API comprises naproxen sodium and/or albendazole benzene sulfonic acid. In further aspects, the hydrated form of the API solvate comprises naproxen sodium dihydrate and/or albendazole benzene sulfonic acid hydrate. In some aspects, the methods further comprise producing the API solvate from an API ansolvate. In some aspects, the methods further comprise removing the excess solvent from the API ansolvate.

In other embodiments, the present disclosure provides methods producing a pharmaceutical formulation comprising: (a) providing starting material comprising a carbamazepine (CBZ) hydrate as an active pharmaceutical ingredient (API); and (b) processing the API by hot melt extrusion (HME) along with at least a first polymer thereby producing a pharmaceutical formulation of an anhydrous form of CBZ, wherein the HME processing is performed at a temperature that is at least 10% less than the temperature required for processing an anhydrous form of CBZ as the starting material. In some aspects, the pharmaceutical formulation is substantially amorphous. In some aspects, the HME is performed using a single screw extruder device. In other aspects, the HME is performed using a twin screw extruder device. In some aspects, the screws of the twin screw extruder device are counter rotating. In other aspects, the screws of the twin screw extruder device are co-rotating. In some aspects, the HME is performed at screw speed of about 50 to 800 rpm. In further aspects, the HME is performed at screw speed of about 50 to 300, 100-300, or 100-200 rpm. In some aspects, the HME is performed using an extruder device having a barrel length/diameter (L/D) ratio of about 20-40:1. In some aspects, the HME processing is performed at a temperature that is at least 15, 20, 25, 30, 35, 40, 45, 50, 55, or 60% less than the temperature required for processing an anhydrous form of CBZ as the starting material. In some aspects, the HME processing is performed at a temperature that is at least 10, 15, 20, 25, 30, 35, 40, 45, 50, 55 or 60° C. less than the temperature required for processing an anhydrous form CBZ as the starting material. In some aspects, the HME processing is performed at a temperature of less than 120° C. In some aspects, the HME processing is performed at a temperature of less than 100° C. In some aspects, the HME processing is performed at a temperature of less than 80° C.

In some aspects, CBZ is present in the pharmaceutical formulation in an amount of from about 0.5% to 20%. In further aspects, CBZ is present in the pharmaceutical formulation in an amount of from about 1% to 15%, 5% to 15%, 7% to 12%, or about 10%. In some aspects, the first polymer comprises a polyvinyl caprolactam-polyvinyl acetate-polyethylene glycol graft copolymer. In some aspects, the pharmaceutical formulation comprises from about 1% to about 90% of the polymer. In further aspects, the pharmaceutical formulation comprises from about 10% to about 90%, about 20% to about 90%, about 30% to about 80%, about 40% to about 70%, or about 50% to about 70% of the polymer. In some aspects, the methods further comprise processing the CBZ by HME along with at least a first polymer and at least a first plasticizer or excipient. In some aspects, the plasticizer is present in in an amount of about 10%-50% w/w. In further aspects, the plasticizer is present in in an amount of about 10%-40%, 20%-50%, or 20%-40% w/w. In some aspects, the first plasticizer comprises vitamin E succinate, propylene glycol or polyethylene glycol. In some aspects, the hydrated form of the CBZ comprises carbamazepine dihydrate. In some aspects, the methods further comprise producing the hydrated form of the CBZ. In further aspects, the methods further comprise removing the excess water the hydrated form of the API.

In still other embodiments, the present disclosure provides substantially amorphous pharmaceutical formulations comprising at least a first API produced by a method of the present disclosure. In some aspects, the formulation is a tablet. In some aspects, the API comprises carbamazepine (CBZ). In some aspects, the formulation comprises a plasticizer. In some aspects, the first polymer is a polyvinyl caprolactam-polyvinyl acetate-polyethylene glycol graft copolymer. In some aspects, the API comprises carbamazepine (CBZ) and the first polymer is a polyvinyl caprolactam-polyvinyl acetate-polyethylene glycol graft copolymer and wherein the formulation comprises vitamin E succinate.

In yet other embodiments, the present disclosure provides substantially amorphous pharmaceutical formulations comprising 5-10% anhydrous carbamazepine (CBZ) and 10%-90% polyvinyl caprolactam-polyvinyl acetate-polyethylene glycol graft copolymer. In some aspects, the formulations further comprise vitamin E succinate. In some aspects, the formulations comprise about 20%-40% vitamin E succinate.

It is contemplated that any embodiment discussed in this specification can be implemented with respect to any method or composition of the invention, and vice versa. Furthermore, compositions of the invention can be used to achieve the methods of the invention.

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate, for example, that a value includes the inherent variation of error for the device, the method being employed to determine the value, or the variation that exists among the study subjects or experimental studies. In the context of this invention, “about” means approximate, and unless otherwise indicated, further means plus/minus 10%.

As used herein, the term “substantially free of” or “substantially free” in terms of a specified component, is used herein to mean that none of the specified component has been purposefully formulated into a composition and/or is present only as a contaminant or in trace amounts. The total amount of all contaminants, by-products, and other material is present in that composition in an amount less than 2%. The term “more substantially free of” or “more substantially free” is used to represent that the composition contains less than 1% of the specific component. The term “essentially free of” or “essentially free” contains less than 0.5% of the specific component.

It will be appreciated that many organic compounds can associate with solvent molecules of the solvent in which they are reacted or from which they are precipitated or crystallized. Compounds associated with solvent molecules are known as “solvates.” Where the solvent is water, the associated is known as a “hydrate.” The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound. Correspondingly, the term “ansolvate” refers to any solid form of a compound wherein the compound is not associated with a solvent molecule. Non-limiting examples of solvates include febuxostat methanol solvate, niclosamide methanol solvate, levofloxacin hemihydrate, cephalexin hydrate, carbamazepine dihydrate, and ampicillin trihydrate, of which the latter four non-limiting examples are more precisely hydrates. The corresponding ansolvates of these non-limiting examples are febuxostat, niclosamide, levofloxacin, cephalexin, carbamazepine, and ampicillin. When an ansolvate is formed as result of the dehydration of the corresponding hydrate, the ansolvate may be referred to as either an ansolvate or more precisely be referred to as an anhydrate.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 shows x-ray powder diffraction (XRPD) spectra of carbamazepine (CBZ) dihydrate, CBZ gamma, and CBZ beta.

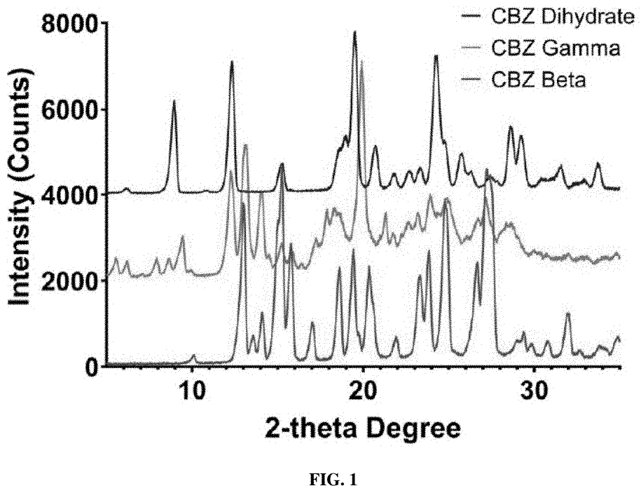

FIG. 2 shows differential scanning calorimetry (DSC) spectrum of carbamazepine dihydrate.

FIG. 3 shows XRPD spectrum of carbamazepine dihydrate undergoing dehydration at various conditions.

FIG. 4 shows polarized light microscope (PLM) images of the dehydration of CBZ dihydrate under hot-stage.

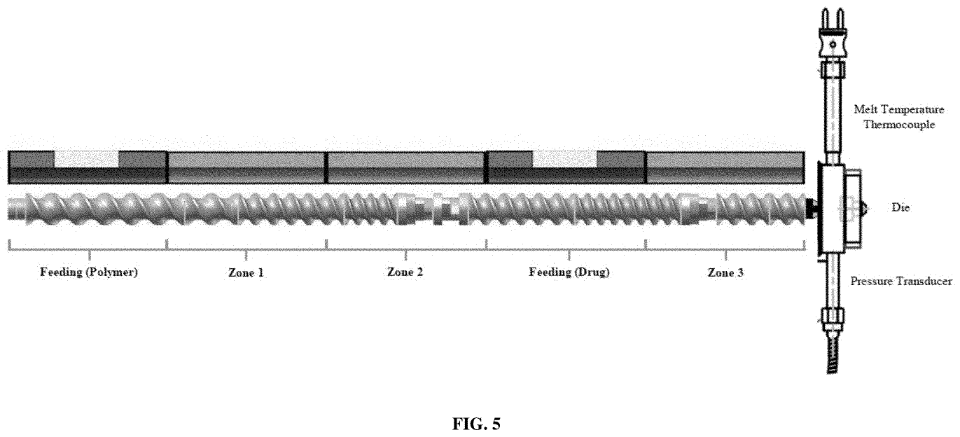

FIG. 5 shows the screw and barrel configuration. All screw elements were 2-flighted; 30°, 60°, and 90° kneading element were used.

FIGS. 6A-6D show comparisons of PXRD between CBZ Soluplus®-Vitamin E succinate ASDs at 10% and 25% w/w drug loading by using CBZ dihydrate (FIGS. 6B & 6D) and CBZ anhydrate (FIGS. 6A & 6C) processed at 60° C. (From left to right, PXRD, PLM, and DSC)

FIGS. 7A-7D shows comparisons of PLM (FIGS. 7A & 7B) and DSC (FIGS. 7C & 7D) between CBZ Soluplus®-Vitamin E succinate ASDs (10% w/w drug loading) by using CBZ dihydrate and CBZ anhydrate processed at 60° C.

FIG. 8 shows a comparison of previous and current screw designs (FIGS. 8A & 8B, respectively). The current screw design has shorter distance from drug feeding port to the kneading element. Higher screw speed can let the drug reach the kneading element faster and higher degree (90°) kneading element results in better mixing.

FIGS. 9A-9F show XPRD spectra, DSC spectra, and PLM images of CBZ dihydrate (FIGS. 9A, 9C, and 9E, respectively) and CBZ anhydrate (FIGS. 9B, 9D, and 9F, respectively) processed at 60° C. CBZ dihydrate formulation exhibited no small crystals under PLM.

FIG. 10 shows a schematic graph of CBZ dihydrate and CBZ anhydrate with different energy states and their correlation to the required extrusion energy.

FIGS. 11A & 11B show PXRD spectra of carbamazepine dihydrate, which was obtained and stayed stable for 3 days at room temperature 56% relative humidity.

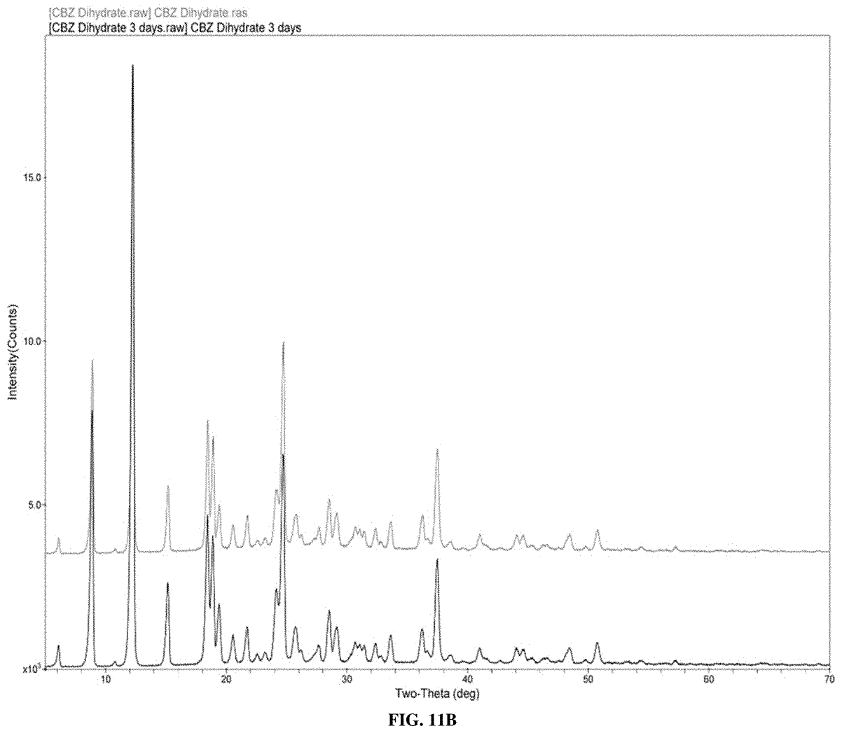

### DESCRIPTION OF ILLUSTRATIVE EMBODIMENTS

In some aspects, the present disclosure provides methods of making pharmaceutical compositions comprising one or more therapeutic agents, one or more pharmaceutically acceptable polymers, and optionally one or more plasticizers. In some embodiments, the methods may be performed under milder conditions, such as lower temperature, and/or may be used in the formulation of temperature sensitive therapeutic agents.

### I. PHARMACEUTICAL COMPOSITIONS

In some aspects, the present disclosure provides pharmaceutical compositions containing a therapeutic agent, a pharmaceutically acceptable polymer, and a mesoporous carrier which have been processed through a thermal process or fusion-based high energy mixing process. In some embodiments, the thermal process may be a hot melt extrusion or a hot melt granulation process. In other embodiments, the fusion-based high energy process is a process which results in an increase in temperature without requiring an external heat input including thermokinetic mixing process such as those described in U.S. Pat. Nos. 8,486,423; 9,339,441; Prasad et al., 2016; LaFountaine et al., 2016; and DiNunzio et al., 2010d. Additionally, these pharmaceutical compositions may show improved solubility or dissolution profiles which result in one or more improved therapeutic parameters or outcomes.

These pharmaceutical compositions may be used and prepared in the absence of a solvent. As used herein a solvent is used within its conventional meaning as a liquid phase component that dissolves one or more components such that those compounds are partially or fully dissolved to form a homogenous mixture.

Additionally, the present pharmaceutical composition may have the added benefit of not requiring the mixing or milling of the components of the composition before being subjected to the thermal or fusion-based high energy processes. Such advantages simplify the formulation process and reduce the possible likelihood of drug decomposition or degradation. In some embodiments, the present compositions may also have the advantage that they allow the processing of the components at a lower temperature to obtain or maintain a lack of crystallinity relative to compositions which contain either the pharmaceutically acceptable polymer or the mesoporous carrier.

A. Therapeutic Agent

The “therapeutic agent” used in the present methods and compositions refers to any substance, compound, drug, medicament, or other primary active ingredient that provides a therapeutic or pharmacological effect when administered to a human or animal. Some non-limiting examples of lipophilic therapeutic agents are BCS classes II and IV compounds or other agents that similarly exhibit poor solubility. The BCS definition describes a compound in which the effective dosing is not soluble in 250 mL of water at a pH from 1-7.5. The USP categories “very slightly soluble” and “insoluble” describe a material that requires 1,000 or more parts of the aqueous liquid to dissolve 1 part solute. As used herein, when a compound is described as poorly soluble, it refers to a compound that has solubility in water of less than 1 mg/mL. In other embodiments, the therapeutic agent is an active agent that has a high melting point. Some non-limiting examples of high melting point therapeutic agents are griseofulvin and theophylline.

When a therapeutically active agent is present in the composition, the therapeutically active agent is present in the composition at a level between 0.5% to 55% w/w, between 1% to 25% w/w, between 5% to 15% w/w, or between 8% to 12% w/w. In some embodiments, the amount of the pharmaceutically acceptable thermoplastic polymer is from about 0.5%, 1%, 5%, 10%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, 50%, to about 55% w/w or any range derivable therein.

Suitable therapeutic agents, including lipophilic therapeutic agents may be any poorly water-soluble, biologically active agents or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal anti-inflammatory drugs (NSAIDS), anthelminthics, antiacne agents, antianginal agents, antiarrhythmic agents, anti-asthma agents, antibacterial agents, anti-benign prostate hypertrophy agents, anticoagulants, anticonvulsants, antidepressants, antidiabetics, antiemetics, antiepileptic s, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the therapeutic agents may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albendazole, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, mebendazole, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the therapeutic agents may be carbamazepine, oxcarbazepine, other anticonvulsant agents, or other members of the general class of azepine compounds. Non-limiting exemplary azepines include a) benzazepines such as benazepril, fenoldopam, GSK-189,254, ivabradine, lorcaserin, semagacestat, and varenicline, b) benzodiazepines such as alprazolam, chlordiazepoxide, clobazam, estazolam, medazepam, midazolam, olanzapine, and triazolam, and c) dibenzazepines such as carbamazepine, clomipramine, clozapine, desipramine, imipramine, lofepramine, mianserin, mirtazapine, opipramol, and trimipramine, and. Other drugs that may be used with this approach include, but are not limited to, hyperthyroid drugs such as carimazole, anticancer agents like cytotoxic agents such as epipodophyllotoxin derivatives, taxanes, bleomycin, anthracyclines, as well as platinum compounds and camptothecin analogs. The following therapeutic agents may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The agents may also include a psychiatric agent such as an antipsychotic, anti-depressive agent, or analgesic and/or tranquilizing agents such as benzodiazepines. The agents may also include a consciousness level-altering agent or an anesthetic agent, such as propofol. The present methods may be used to prepare pharmaceutical compositions with the appropriate pharmacokinetic properties for use as therapeutics.

In some aspects, the method may be most used with materials that undergo degradation at an elevated temperature or pressure. The therapeutic agents that may be used include those which decompose at a temperature above about 50° C. In some embodiments, the therapeutic agent decomposes above a temperature of 80° C. In some embodiments, the therapeutic agent decomposes above a temperature of 100° C. In some embodiments, the therapeutic agent decomposes above a temperature of 150° C. The therapeutic agent that may be used include therein which decompose at a temperature of greater than about 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., or 150° C.

B. Pharmaceutically Acceptable Polymers

In some aspects, the present disclosure provides compositions which may further comprise a pharmaceutically acceptable polymer. In some embodiments, the polymer has been approved for use in a pharmaceutical formulation and is known to undergo softening or increased pliability when raised above a specific temperature without substantially degrading. Additionally, the pharmaceutically acceptable polymer may also be known to enhance the dissolution of one or more of the therapeutic agents in the composition or pharmaceutical composition.

When a pharmaceutically acceptable polymer is present in the composition, the pharmaceutically acceptable polymer is present in the composition at a level between about 1% to about 49% w/w, between about 5% to about 45% w/w, between about 10% to about 40% w/w, between about 20% to about 40% w/w, between about 20% to about 30% w/w of the total pharmaceutical composition or the total composition. In some embodiments, the amount of the pharmaceutically acceptable polymer is from about 1%, 5%, 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, 48%, to about 49% w/w or any range derivable therein.

In some aspects, Flory-Huggins theory can be used as a preformulation test to guide or predict appropriate therapeutic agent and pharmaceutically acceptable polymer combination. Flory-Huggins theory may be used to predict miscibility information for amorphous drug-polymer systems by evaluating the drug-polymer interaction parameter, χ, to calculate the free energy of mixing (ΔGₘᵢₓ) for the system. The χ value stems from the non-ideal entropy of mixing of the pharmaceutically acceptable polymer molecule with the therapeutic agent and takes into account the contribution due to the enthalpy of mixing (Bansal et al., 2016). More negative χ values predict miscibility whereas more positive χ values predict immiscibility of the therapeutic agent-polymer system (Bansal et al., 2016; Marsac et al., 2006). According to Flory-Huggins theory,

$$Δ {G}_{mix} = RT ( {Φ}_{drug} ln {Φ}_{drug} + \frac{{Φ}_{polymer}}{m} ln {Φ}_{polymer} + {χΦ}_{drug} {Φ}_{polymer} ) \qquad ( Equation 1 )$$

where Φ is the volume fraction, χ is the Flory-Huggins interaction parameter, R is the molar gas constant, and T is the temperature. m is the ratio of the volume of a pharmaceutically acceptable polymer to the therapeutic agent molecular volume and,

$$m = \frac{{MW}_{polymer} \text{/} {ρ}_{polymer}}{{MW}_{drug} \text{/} {ρ}_{drug}} \qquad ( Equation 2 )$$

where the MW_{polymer} and MW_{drug} are the molecular weight of the pharmaceutically acceptable polymer and therapeutic agent, respectively, and the ρ_{polymer} and ρ_{drug} are the density of pharmaceutically acceptable polymer and therapeutic agent, respectively. The primary method for determining the χ value is by analyzing the melting point depression of the solid dispersion system, which is often, analyzed using differential scanning calorimetry (DSC). DSC is utilized to determine the melting point onset (Zhao et al., 2011), melting temperature (Lin and Huang, 2010; Marsac et al., 2008), or melt endpoint (Tian et al., 2013). Following analysis of melting point depressions, the χ value can be calculated using the following rearranged equation (Marsac et al., 2006).

$$( \frac{1}{{T}_{M}^{mix}} - \frac{1}{{T}_{M}^{pure}} ) ( \frac{Δ {H}_{fus}}{- R} ) - ln {Φ}_{drug} - ( 1 - \frac{1}{m} ) {Φ}_{polymer} = {χΦ}_{polymer}^{2} \qquad ( Equation 3 )$$

where T_{M} values are the melting points of the mixture of pure therapeutic agent, R is the ideal gas constant, ΔH_{fus} is the heat of fusion for the pure therapeutic agent, m is a constant for the relative size of the pharmaceutically acceptable polymer to the therapeutic agent, and the Φ values are volume fraction of therapeutic agent or pharmaceutically acceptable polymer. If the plot of the left side of the rearranged equation vs. the Φ² value for the pharmaceutically acceptable polymer demonstrates linearity, the slope of the best-fit line is considered to be equivalent to χ. By understanding χ as a function of temperature, metastable and unstable regions for the combination can be predicted by generating a spinodal (boundary between unstable and metastable regions) and binodal (boundary between metastable and stable regions) curves (Huang et al., 2016). If the solid dispersion system's components are stable, these systems tend to remain in a single-phase, while metastable and unstable systems tend to phase separate into drug-rich and polymer-rich domains upon storage. Without wishing to be bound by any theory it is believed that the tendency to recrystallize occurs because the high-energy amorphous state is generally unstable (Marsac et al., 2010; Purohit and Taylor, 2015). Therefore, in some embodiments, Flory-Huggins theory as a preformulation test contemplates that the combination of the pharmaceutically acceptable polymer and the therapeutic agent exhibits a stable combination. In other aspects, the present combinations of the pharmaceutically acceptable polymer and the therapeutic agent exhibits a positive χ value.

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups, which are charged at a physiologically relevant pH. Some non-limiting examples of neutral non-cellulosic polymers include polyvinyl pyrrolidone, polyvinyl alcohol, copovidone, poloxamer, polyethylene oxide, polypropylene oxide, polyvinylpyrrolidone-co-vinylacetate, polyethylene, polycaprolactone, and polyethylene-co-polypropylene. Some examples of ionizable non-celluolosic polymers include polymethacrylate or polyacrylate such as Eudragit®. Some non-limiting examples of ionizable cellulosic polymers include hydroxyalkylalkyl cellulose ester such as cellulose acetate phthalate and hydroxypropyl methyl cellulose acetate succinate, carboxyalkyl cellulose such as carboxymethyl cellulose and alkali metal salts thereof, such as sodium salts, and carboxyalkylalkyl cellulose including carboxymethylethyl cellulose, carboxyalkyl cellulose ester such as carboxymethyl cellulose butyrate, carboxymethyl cellulose propionate, carboxymethyl cellulose acetate butyrate, and carboxymethyl cellulose acetate propionate. Finally, some non-limiting examples of neutral cellulosic polymers include alkylcelluloses such as methylcellulose, hydroxyalkylcelluloses such as hydroxymethylcellulose, hydroxypropyl cellulose, hydroxyethylcellulose, and hydroxybutylcellulose, hydroxyalkyl alkylcelluloses such as hydroxyethyl methylcellulose and hydroxypropyl methyl cellulose, starches, pectins, chitosan or chitin and copolymers and mixtures thereof, and polysaccharides such as tragacanth, gum arabic, guar gum, and xanthan gum.

Some specific pharmaceutically acceptable polymers which may be used include, for example, Eudragit™ RS PO, Eudragit™ S100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate-methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate-co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate-co-methyl methacrylate-co-trimethylammonioethyl methacrylate chloride 1:2:0.1), Eudragit L-30-D™ (MA-EA, 1:1), Eudragit L-100-55™ (MA-EA, 1:1), hydroxypropylmethylcellulose acetate succinate (HPMCAS), polyvinyl caprolactam-polyvinyl acetate-PEG graft copolymer (SOLUPLUS®), polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, xanthan gum, Affinisol™ HPMC HME, Eudragit EPO, and Soluplus, Copovidone. Other excipients which may be used include wetting agents/surfactants including tweens, sodium lauryl sulfate and the like, disintegrants including superdisintegrants including crospovidone, sodium starch glycolate, and the like, carriers including microcrystalline cellulose, carbohydrates like lactose, mannitol and the like, anti-tacking agents like magnesium stearate, stearic acid, talc, silicone dioxide and the like.

### II. THERMAL METHODS

Thus, in one aspect, the present disclosure provides pharmaceutical compositions which may be prepared using a thermal or fusion-based high energy process. Such process may include hot melt extrusion, hot melt granulation, melt mixing, spray congealing, sintering/curing, injection molding, or a thermokinetic mixing process such as the KinetiSol method. Similar thermal processing methods are described in LaFountaine et al., 2016a, Keen et al., 2013, Vynckier et al., 2014, Lang et al., 2014, Repka et al., 2007, Crowley et al., 2007, DiNunzio et al., 2010a, DiNunzio et al., 2010b, DiNunzio et al., 2010c, DiNunzio et al., 2010d, Hughey et al., 2010, Hughey et al., 2011, LaFountaine et al., 2016b, and Prasad et al., 2016, all of which are incorporated herein by reference. In some embodiments of these present disclosure, the pharmaceutical compositions may be prepared using a thermal process such as hot melt extrusion or hot melt granulation. In other embodiments, a fusion based process including thermokinetic mixing process such as those described at least in U.S. Pat. Nos. 8,486,423 and 9,339,440, the entire contents of which are herein incorporated by reference.

A non-limiting list of instruments which may be used to thermally process the pharmaceutical compositions described herein include hot melt extruders available from ThermoFisher, such as a minilab compounder, or Leistritz, such as a twin-screw extruder. Alternatively, a fusion-based high energy process instrument that does not require external heat input, including such as a thermokinetic mixer as described in U.S. Pat. Nos. 8,486,423 and 9,339,440 may be used to process the pharmaceutical composition.

In some aspects, the extruder may comprise heating the composition to a temperature from about 30° C. to about 250° C. In some embodiments, the temperature is from about 45° C. to about 150° C. The temperature that may be used is from about 50° C., 60° C., 65° C., 70° C., 75° C., 80° C., 90° C., 92° C., 94° C., 96° C., 98° C., 100° C., 102° C., 104° C., 106° C., 108° C., 110° C., 112° C., 114° C., 116° C., 118° C., 120° C., 125° C., 130° C., 135° C., 140° C., 145° C., 150° C., 155° C., 160° C., 165° C., 170° C., 175° C., 180° C., 190° C., 200° C., 225° C., to about 250° C. or any range derivable therein.

The extrudate produced following the extrusion process will generally comprise the therapeutic agent, the pharmaceutically acceptable polymer, and optionally a plasticizer. A plasticizer may be useful to lower the glass transition temperature of the composition. A non-limiting example of a plasticizer useful in the methods disclosed herein is vitamin E succinate, propylene glycol, polyethylene glycol (e.g., 300, 400, 600), and the like. Further examples of plasticizers include, but are not limited to Triethyl citrate, Dibutyl, Triethyl citrate (TEC), Tributyl citrate (TBC), Acetyl triethyl citrate (ATEC), Dibutyl sebacate (DBS) Diethyl phthalate (DEP), Dibutyl phthalate (DBP) Diesters and triesters of alcohols, Triacetin (TA), Vegetable oils, Fractionated coconut oil, Acetylated monoglycerides, PEG8000, TPGS, PEG/TPGS mixtures and triethyl citrate. The extrudate may be in the form of granules of a desired mesh size or diameter, rods that can be cut and shaped into tablets, and films of a suitable thickness that shaped forms can be punched into suitable size and shape for administration. This extrudate may be used in further processing steps to yield the final pharmaceutical product or composition. The extrudate of the pharmaceutical composition may be dried, formed, milled, sieved, or any combination of these processes to obtain a final composition which may be administered to a patient. Such processes are routine and known in the art and include formulating the specific product to obtain a final pharmaceutical or nutraceutical product. Additionally, the extrudate of the pharmaceutical composition obtained may be processed using a tablet press to obtain a final table. Additionally, it may be milled and combined with one or more additional excipients to form a capsule or pressed into a tablet. The resultant pharmaceutical composition may also be dissolved in a pharmaceutically acceptable solvent to obtain a syrup, a suspension, an emulsion, or a solution.

### III. EXAMPLES

To facilitate a better understanding of the present invention, the following examples of specific embodiments are given. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. In no way should the following examples be read to limit or define the entire scope of the invention.

### Example 1—Preparation of Drug Hydrate/Solvate and Characterization

Materials. Carbamazepine (CBZ; Letco Medical, Decatur, Ala.) was selected as the drug. Soluplus® was purchased from BASF (Florham Park, N.J.). Vitamin E Succinate was purchased from VWR International, LLC. (Radnor, Pa.). All chemicals used were ACS reagent. Chemicals were of reagent grade.

Methods. Carbamazepine Dihydrate Preparation: CBZ anhydrate was suspended in water at ambient condition for 24 hours. The suspension was filtered by vacuum filtration, and the resulting slurry was lyophilized at 0° C. and 2500 mTorr for 18 hours to obtain CBZ dihydrate.

Preparation of Blends: Physical blends containing 30% w/w Vitamin E Succinate and 70% w/w Soluplus® were prepared using a Turbula mixer (Glen Mills Inc., Clifton, N.J., USA).

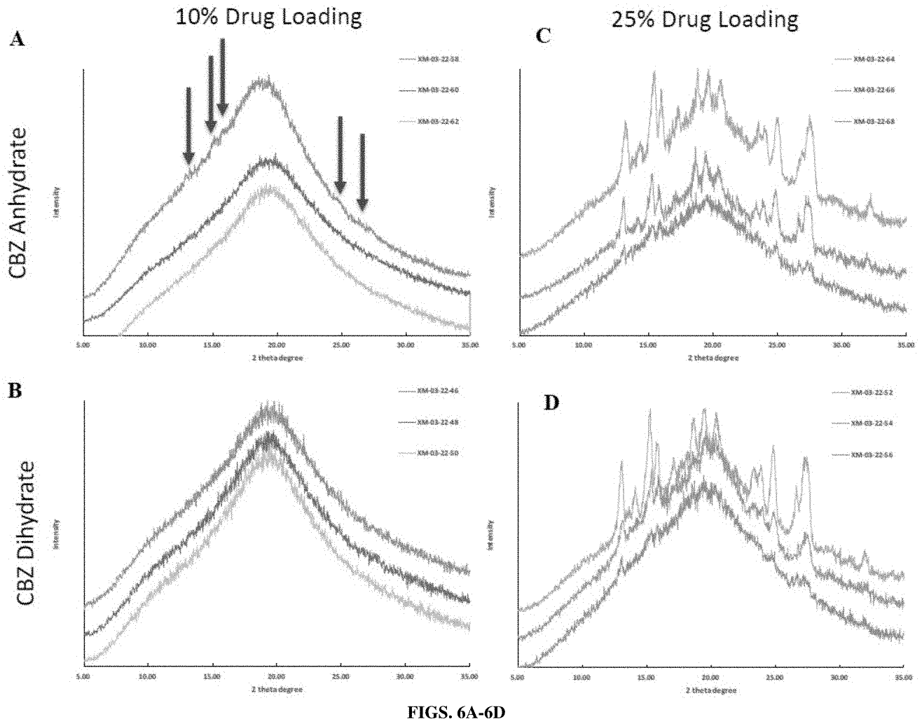

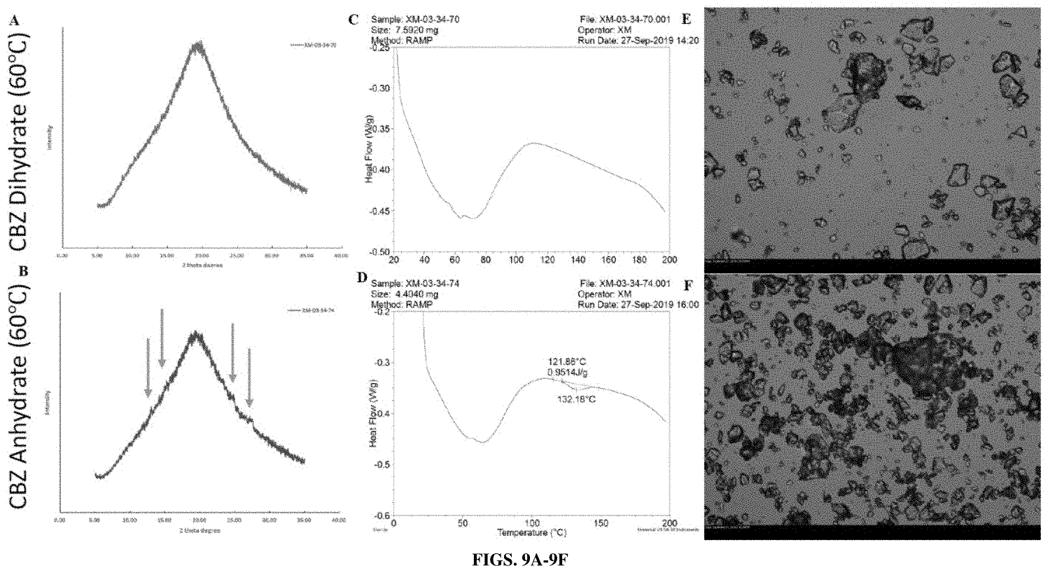

Production of Extrudates: A co-rotating Leistritz Micro-18 twin-screw extruder (American Leistritz Extruder Corp., Somerville, N.J., USA) was used to prepare carbamazepine ASDs. The extruder barrel configuration is shown in FIG. 5. The extruder was set up with six sections (feeding zone for polymer, zone 1, zone 2, feeding zone for drug, zone 3, and endplate/die). The feeding zone for polymer was water cooled, and the temperature was maintained below 35° C. throughout experiments. All other sections were maintained at various elevated temperatures depending on the particular extrusion being performed. Two twin-screw volumetric feeders (Brabender Technology, Duisburg, Germany) were used to feed the drug (CBZ anhydrate or CBZ dihydrate) and physical blends of Soluplus® and Vitamin E Succinate at the feeding zone for drug and feeding zone for polymer, respectively. Two screw designs were used to prepare CBZ ASDs as shown in FIG. 8. CBZ extrudates were prepared under various barrel temperatures (ranging from 60-140° C.) and at different screw speeds (100 and 200 rpm). Extrudates were cooled at room temperature. Tables 1 and 2 show parameters that were varied during an extrusion study using screw design 1 and screw design 2, respectively.

TABLE 1
Extrusion runs and conditions using screw design 1.

| | | | Drug | | Polymer | |
|---|---|---|---|---|---|---|
| Lot # | Run | Drug | Content w/w | Polymer | Content w/w | Plasticizer |
| XM-03-22-58 | 58 | CBZ Raw | 10% | Soluplus | 70% in | Vitamin E |
| XM-03-22-60 | 60 | | | | polymer blends | Succinate |
| XM-03-22-62 | 62 | | | | | |
| XM-03-22-64 | 64 | | 25% | | | |
| XM-03-22-66 | 66 | | | | | |
| XM-03-22-68 | 68 | | | | | |
| XM-03-22-46 | 46 | CBZ Dihydrate | 10% | Soluplus | 70% in | Vitamin E |
| XM-03-22-48 | 48 | | | | polymer blends | Succinate |
| XM-03-22-50 | 50 | | | | | |
| XM-03-22-52 | 52 | | 25% | | | |
| XM-03-22-54 | 54 | | | | | |
| XM-03-22-56 | 56 | | | | | |

| | | | Temperature | |
|---|---|---|---|---|
| | | | (zone 1, zone 2, | |
| | Plasticizer | | feeding zone for drug, | Screw |
| Lot # | Content w/w | Feed Rate g/min | zone 3, endplate, die) | Speed rpm |
| XM-03-22-58 | 30% in | Total 6 | 140-140-60-60-60-60 | 100 |
| XM-03-22-60 | polymer blends | | 140-140-120-120-120-120 | |
| XM-03-22-62 | | | 140 | |
| XM-03-22-64 | | | 140-140-60-60-60-60 | |
| XM-03-22-66 | | | 140-140-120-120-120-120 | |
| XM-03-22-68 | | | 140 | |
| XM-03-22-46 | 30% in | Total 6 | 140-140-60-60-60-60 | |
| XM-03-22-48 | polymer blends | | 140-140-120-120-120-120 | |
| XM-03-22-50 | | | 140 | |
| XM-03-22-52 | | | 140-140-60-60-60-60 | |
| XM-03-22-54 | | | 140-140-120-120-120-120 | |
| XM-03-22-56 | | | 140 | |

TABLE 2
Extrusion runs and conditions using screw design 2.

| | | | Drug | | Polymer | | Plasticizer |
|---|---|---|---|---|---|---|---|
| Lot # | Run | Drug | Content w/w | Polymer | Content w/w | Plasticizer | Content w/w |
| XM-03-34-70 | 70 | CBZ Dihydrate | 10% | Soluplus | 70% in | Vitamin E | 30% in |
| XM-03-34-74 | 74 | CBZ Raw | | | polymer blends | Succinate | polymer blends |
| XM-03-34-75 | 75 | | | | | | |
| XM-03-34-76 | 76 | | | | | | |

| | | Temperature | | |
|---|---|---|---|---|
| | | (zone 1, zone 2, | Screw | |
| | Feed Rate | feeding zone for drug, | Speed | |
| Lot # | g/min | zone 3, endplate, die) | rpm | Appearance |
| XM-03-34-70 | Total 6 | 140-140-60-60-60-60 | 200 | Transparent |
| XM-03-34-74 | | 140-140-60-60-60-60 | | Opaque |
| XM-03-34-75 | | 140-140-100-100-100-100 | | |
| XM-03-34-76 | | 140-140-140-140-140-140 | | |

Differential Scanning calorimetry (DSC): Conventional DSC and modulated DSC (mDSC) were performed to characterize the physical mixtures and extrudates. All samples were analyzed using a TA Instruments Model Auto Q20 DSC (TA Instruments, New Castle, Del., USA) under a dry nitrogen purge (200 mL/min) with the RCS40 (TA Instruments, New Castle, Del., USA) refrigerated cooling system accessory. Samples were weighed accurately (5-6 mg) in Tzero® (TA Instruments, New Castle, Del., USA) pans and crimped with a pinhole aluminum lid. Conventional DSC was used to determine the melting enthalpy of CBZ and the melting temperature of physical mixtures and all extrudates. 10° C./min up to 200° C. was set for conventional DSC. A modulation of ±1° C. every 60 s and heating rate of 3° C./min up to 200° C. were applied for mDSC.

Powder X-ray diffraction (PXRD): PXRD was used to measure the crystallinity of extrudates. We used a Rigaku Miniflex 600 (Rigaku, Tokyo, Japan) X-Ray Diffractometer equipped with Cu Kα radiation at 40 kV and 15 mA. Data were collected in a scan-spin mode with a step size of 0.02° and a scan speed of 1°/min over a 20 range of 5-35°. Data were analyzed using Jade 9 software (KS Analytical Systems, Aubrey, Tex.).

Polarized Light Microscopy (PLM): PLM equipped with a hot stage was used to visualize and characterize the presence of crystalline drug particles inside the extrudates. PLM studies were conducted on a Olumpus polarizing optical microscope (Olympus Life Science, Waltham, Mass.). The milled extrudates were placed between a glass slide and a cover glass and then fixed on Linkham THMS600 hot stage (Linkham Scientific Instruments Ltd., Surry, England).

### Example 2—Compositions Made According to the Embodiments

It was hypothesized that the use of hydrated APIs in hot-melt extrusion (HME) may result in milder processing parameters. Carbamazepine (CBZ) was chosen as the model drug (dehydrate and anhydrate). Soluplus® was selected as the matrix excipient based on its relatively low glass transition temperature (72° C.), suitable viscosity, and suitable miscibility with CBZ. Structures and properties of carbamazepine and various matrix excipients are shown below in Schemes 1 and 2.

Scheme 1 Structure of carbamazepine

| Property | Value |
|---|---|
| BCS Class | II |
| Aq. Solubility | 113 μg/mL |
| pKa | 13.4 |
| Log P | 2.45 |
| T_{dehydration} | 20-80° C. |
| T_{melt(anhydrate)} | 175, 189° C. |

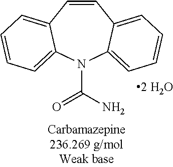 [SMILES: NC(=O)N1c2ccccc2C=Cc2ccccc21]

Scheme 2 Structures of various matrix excipients and associated T_{g}values.

| Name | Property |
|---|---|
| Kollidon ® VA6₄ (PVP VA6₄) | T_{g} = 105° C. |
| Affinisol ™ HPMC HME | T_{g} = 103° C. |
| Soluplus ® | T_{g} = 72° C. |
| Eudragit ® EPO | T_{g} = 45° C. |

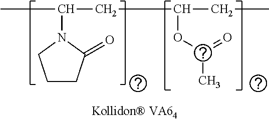

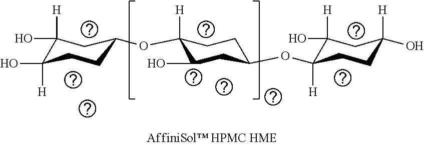

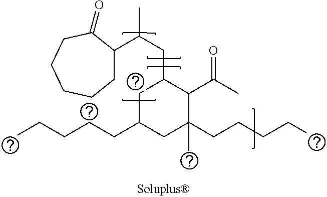

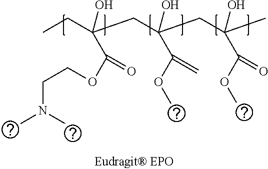

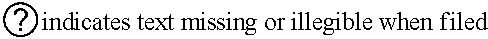

In order to extrude the drug and polymer blends at a low temperature, 30% w/w Vitamin E succinate (VeS) was added and mixed with Soluplus®. CBZ dihydrate was prepared by suspending CBZ anhydrate in water for 24 hours. The suspension was filtered by vacuum filtration, and the resulting slurry was lyophilized at 0° C. and 2500 mTorr for 18 hours. Other methods for drying CBZ dihydrate were tested, including but not limited to freeze-drying. Differential scanning calorimetry (DSC), powder X-ray diffraction (PXRD), and polarized light microscopy (PLM) were used to verify the successful formation of the carbamazepine dihydrate and to quantitate the crystallinity in the resulting extrudates. Carbamazepine dihydrate was obtained and stayed stable under 56% relative humidity at room temperature throughout the whole experiments. In addition, the dehydration of CBZ dihydrate at various temperatures was studied using DSC and PXRD. Formulations with CBZ dihydrate were processed using a Leistritz Micro-18 extruder with a side feeding barrel configuration, different drug loadings, and various barrel temperatures. Also, formulations with CBZ anhydrate were processed at the same extrusion conditions to investigate the influence of CBZ dihydrate on the preparation of CBZ ASDs.

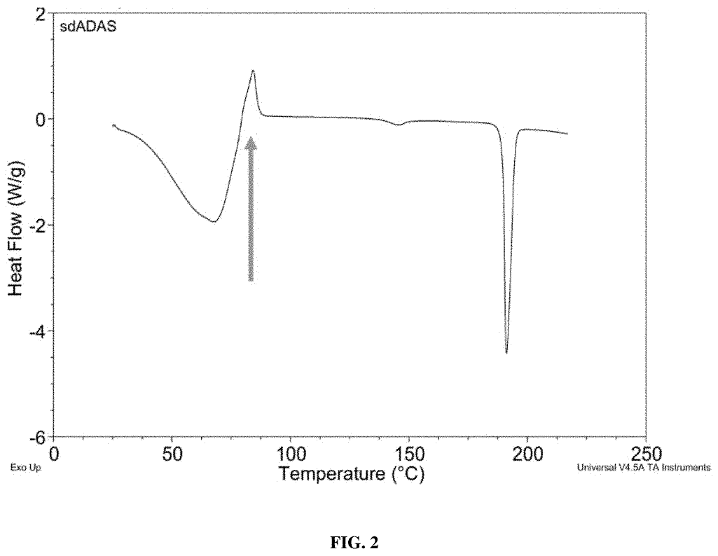

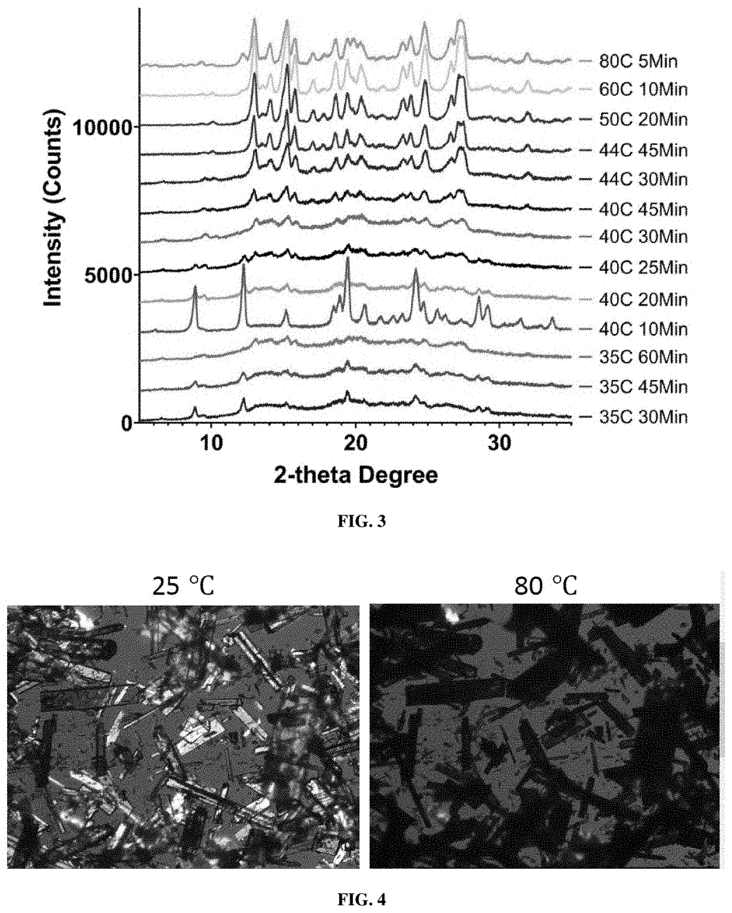

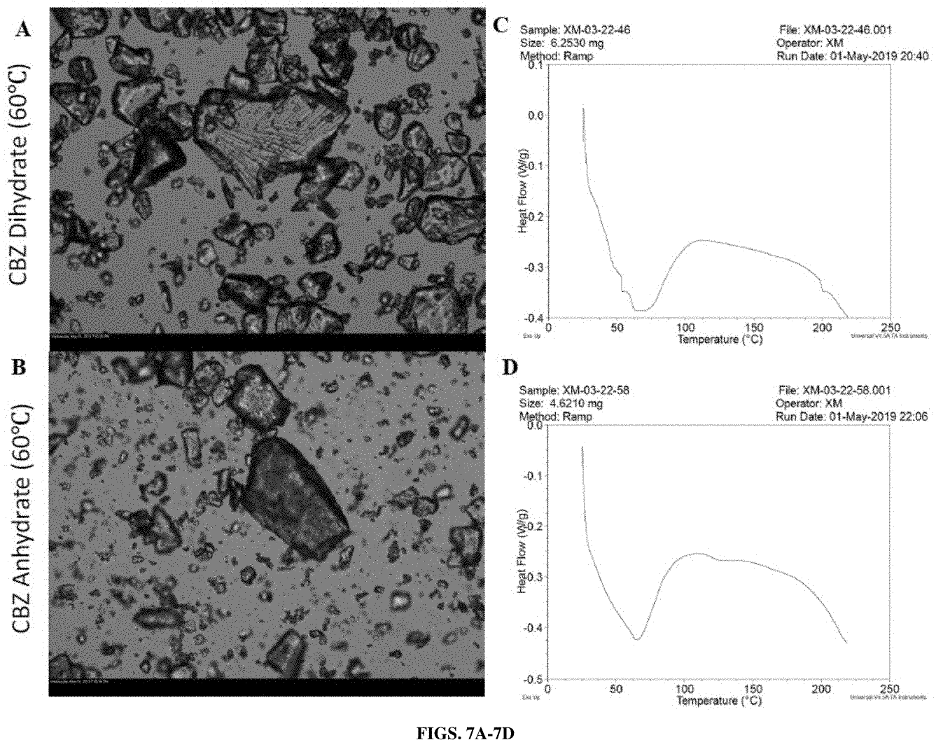

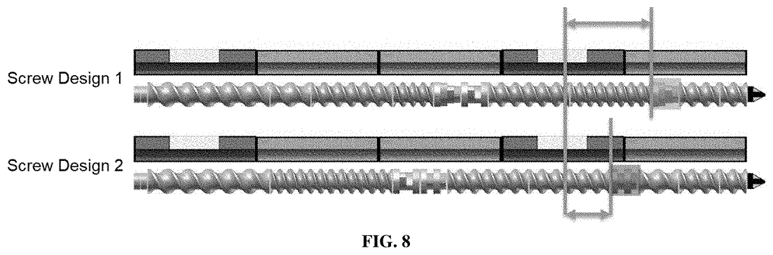

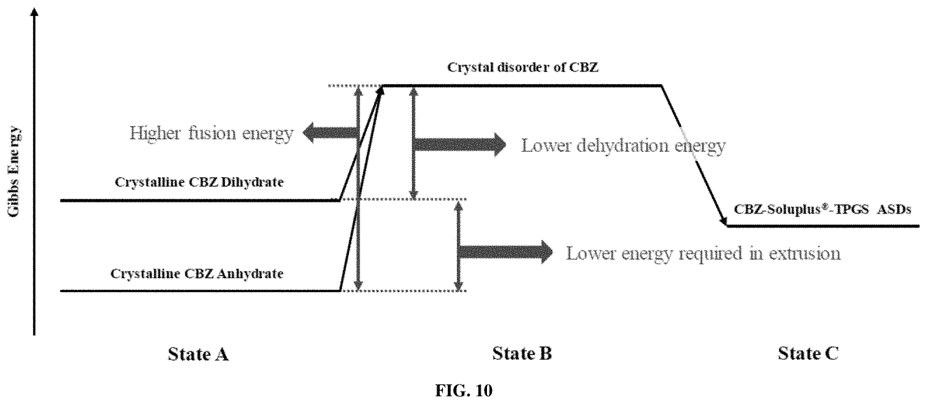

Dehydration/crystal disorder of CBZ dihydrate occurred at 40 to 70° C. followed by recrystallization of the disordered CBZ molecules at 86° C. as shown in FIG. 2. At different temperatures (e.g., 35, 40, 44, 50, 60, and 80° C.), CBZ dihydrate converted into different polymorphs depending on the duration time. It was found that at 35° C. for 60 mins or 40° C. for 30 mins, CBZ dihydrate converted into a disordered state with the minimum presence of recrystallized beta polymorph as shown in FIG. 3. In order to utilize this higher energy disorder state and to minimize the occurrence of recrystallization (only enabling the dehydration of CBZ dihydrate), polymer blends should melt before the side feeding of the drug. Therefore, extrusion temperatures were set from 60° C. to 140° C. to assure the polymer blends are in the molten state at the side feeding zone and still exhibit extrudable viscosity. A side feeding barrel configuration was used in this study. For screw designs 1 and 2, by using CBZ dihydrate, CBZ Soluplus®-VeS ASDs (10% w/w drug loading) were successfully obtained at all three temperatures, 60, 120, and 140° C. confirmed by PLM and DSC as shown in FIG. 6-9, while the ASDs of using CBZ anhydrate were only obtained above 120° C. However, at 25% w/w drug loading, CBZ Soluplus®-VeS ASDs cannot be achieved at those three temperatures either by using CBZ dihydrate or CBZ anhydrate. In addition, screw design 2 resulted in less crystallinity for the extrudates prepared using CBZ dihydrate compared to screw design 1. This was due to more intense mixing and short residence time between drug feeding zone and mixing elements.

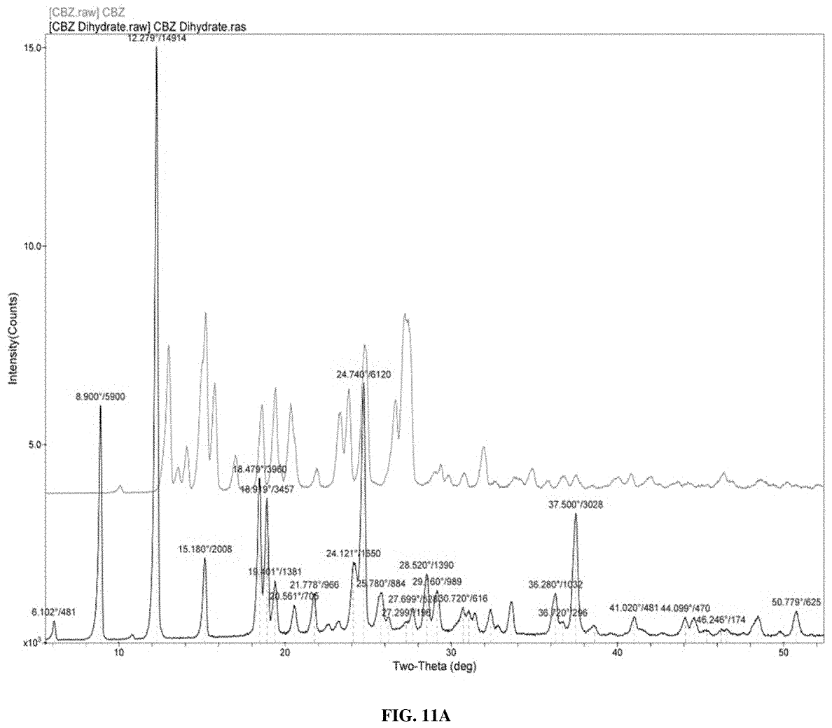

Compared to CBZ anhydrate, the required extrusion temperature to achieve CBZ Soluplus®-VeS ASDs (10% w/w drug loading) was successfully lowered from 120° C. to 60° C. with the help of CBZ dihydrate. The present disclosure shows that proper utilization of higher drug energy states caused by form change during the extrusion process can significantly expand the HME design space as shown in FIG. 10. The extrusion temperature window is limited to the viscosity of the polymer and the recrystallization tendency of the disordered drug molecules.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bertilsson, Clin. Pharmacokinetics, 3(2):128-143, 1978.
Han and Suryanarayanan, Pharm. Develop. Technol., 3(4):587-596, 1998.
Huang et al., AAPS PharmSciTech, 17(1):106-119, 2016.
Li et al., Pharm. Develop. Technol., 5(2):257-266, 2000.
Sethia and Squillante, J. Pharm. Sci., 91(9):1948-1957, 2002.
Surana et al., Pharm. Res., 21(7):1167-1176, 2004.

1. A method producing a pharmaceutical formulation comprising:
(A) providing starting material comprising an active pharmaceutical ingredient (API) solvate; and
(B) processing the API solvate by hot melt extrusion (HME) along with at least a first polymer thereby producing a pharmaceutical formulation of the API ansolvate,
wherein the HME processing is performed at a temperature that is at least 10% less than the temperature required for processing the API ansolvate as the starting material.

2. The method of claim 1, wherein the API solvate is an API hydrate.

3. The method of claim 1, wherein the pharmaceutical formulation is substantially amorphous.

4.-7. (canceled)

8. The method of claim 1, wherein the HME is performed at screw speed of about 50 to 800 rpm.

9.-11. (canceled)

12. The method of claim 1, wherein the HME processing is performed at a temperature that is at least 10° C., 15° C., 20° C., 25° C., 30° C., 35° C., 40° C., 45° C., 50° C., 55° C. or 60° C. less than the temperature required for processing an anhydrous form of the API as the starting material.

13.-15. (canceled)

16. The method of claim 1, wherein the API solvate is a monosolvate or disolvate.

17.-24. (canceled)

24. The method of claim 1, wherein the API is present in the pharmaceutical formulation in an amount of from about 0.5% to 20%.

25.-26. (canceled)

27. The method of claim 1, wherein the pharmaceutical formulation comprises from about 1% to about 90% of the polymer.

28. (canceled)

29. The method of claim 1, further comprising processing the API by HME along with at least a first polymer and at least a first plasticizer or excipient.

30.-32. (canceled)

33. The method of claim 1, wherein the API comprises carbamazepine (CBZ), naproxen sodium, or albendazole benzene sulfonic acid.

34.-38. (canceled)

39. A method producing a pharmaceutical formulation comprising:
(A) providing starting material comprising a carbamazepine (CBZ) hydrate as an active pharmaceutical ingredient (API);
(B) processing the API by hot melt extrusion (HME) along with at least a first polymer thereby producing a pharmaceutical formulation of an anhydrous form of CBZ,
wherein the HME processing is performed at a temperature that is at least 10% less than the temperature required for processing an anhydrous form of CBZ as the starting material.

40. The method of claim 39, wherein the pharmaceutical formulation is substantially amorphous.

41.-48. (canceled)

49. The method of claim 39, wherein the HME processing is performed at a temperature that is at least 10, 15, 20, 25, 30, 35, 40, 45, 50, 55, or 60° C. less than the temperature required for processing an anhydrous form CBZ as the starting material.

50. The method of claim 39, wherein the HME processing is performed at a temperature of less than 120° C.

51.-52. (canceled)

53. The method of claim 39, wherein CBZ is present in the pharmaceutical formulation in an amount of from about 0.5% to 20%.

54.-55. (canceled)

56. The method of claim 39, wherein the pharmaceutical formulation comprises from about 1% to about 90% of the polymer.

57. (canceled)

58. The method of claim 39, further comprising processing the CBZ by HME along with at least a first polymer and at least a first plasticizer or excipient.

59.-61. (canceled)

62. The method of claim 33, wherein the hydrated form of the CBZ comprises carbamazepine dihydrate.

63.-64. (canceled)

65. A substantially amorphous pharmaceutical formulation comprising at least a first API produced by a method of claim 1.

66.-70. (canceled)

71. A substantially amorphous pharmaceutical formulation comprising 5-10% anhydrous carbamazepine (CBZ) and 10%-90% polyvinyl caprolactam-polyvinyl acetate-polyethylene glycol graft copolymer.

72.-74. (canceled)
